(19) United States
(12) Patent Application Publication
(10) Pub. No.: US 2026/0325700 A1
(43) Pub. Date: Oct. 22, 2026

(54) AIR DISINFECTION SYSTEM FOR DISINFECTING ELEVATOR CABIN

(71) Applicant: Mirza Faizan, Plano, TX (US)

(72) Inventors: Mirza Faizan, Plano, TX (US); Reva Agrahari, Plano, TX (US); Aybach Charkas, Plano, TX (US); Mishaal Ayesha Umar, Plano, TX (US); Arini Kiran, Plano, TX (US); Aaron Jacob, Plano, TX (US); Akansh Karthik, Plano, TX (US); Francis Paul Kurian, Plano, TX (US); Rahil Kakar, Plano, TX (US); Arjun Sriram, Plano, TX (US)

(21) Appl. No.: 19/183,883

(22) Filed: Apr. 20, 2025

(51) Int. Cl. A61L 9/20

(52) U.S. Cl. CPC A61L 9/20; A61L 2209/111; A61L 2209/14

(57) ABSTRACT

An air disinfection system for disinfecting a cabin of an elevator is disclosed. The air disinfection system comprises a set of panels, a vacuum box, a vacuum pump to create negative pressure in the vacuum box, an air disinfection chamber, a plurality of perforations in each panel, a plurality of valves in each panel, a plurality of sensors to detect a sneeze of a user in the cabin, and a processor that controls a set of valves of the plurality of valves to open a set of perforations of the plurality of perforations, control the vacuum box to create negative pressure to suck infected air from the specific portion of cabin via the opened set of perforations, control the air disinfection chamber to disinfect the sucked infected air, control a first valve of the air disinfection chamber to allow passage of the disinfected air out of the air disinfection chamber.

### DESCRIPTION

### Technical Field

This disclosure relates generally to an air disinfection system, and more particularly to a UV-C light sterilization system to disinfect infected air in elevators in and out of use.

### Background

Elevator cabins typically have minimal fresh air intake and rely on mechanical ventilation systems that may not effectively filter out airborne pathogens. This limited air exchange can lead to the accumulation of bio aerosols, which can carry viruses and bacteria for extended periods, increasing the risk of airborne disease transmission among patients.

Pathogen droplets and aerosols can remain viable in enclosed spaces like elevator cabins for considerable time (up to 18 minutes), allowing new passengers to inhale potentially infectious aerosols from previous occupants. This persistence complicates disinfection efforts, as the air must be continuously monitored and treated to ensure safety.

Current ventilation designs often do not adequately address the dynamics of airflow and droplet dispersion within confined spaces. The placement of air inlets and outlets significantly influences airflow patterns, which can either exacerbate or mitigate the spread of contaminants. Without proper design considerations, even advanced air purification technologies may fail to eliminate airborne transmission risks. Ensuring complete coverage of the cabin to prevent shadowing effects where some areas remain untreated.

### SUMMARY OF INVENTION

In an embodiment, an air disinfection system for disinfecting a cabin of an elevator is disclosed. The system includes a set of panels, wherein each panel of the set of panels is mounted on a corresponding wall of the set of walls of the cabin. The system further includes a vacuum box and a vacuum pump configured to create negative pressure in the vacuum box. The set of panels further includes a plurality of perforations in each panel, a plurality of valves in each panel, wherein each valve of the plurality of valves controls opening and closing of a corresponding perforation of the plurality of perforations. The system further includes a plurality of sensors configured to detect a sneeze of a user in the cabin and a processor which is configured to control, based on the detection of the sneeze of the user in a specific portion of the cabin, a set of valves of the plurality of valves to open a corresponding set of perforations of the plurality of perforations, wherein the set of perforations are within the specific portion of the cabin in which sneeze is detected. The processor further controls the vacuum box to create negative pressure to suck infected air from the specific portion of cabin via the opened set of perforations, wherein the sucked infected air is transferred to the air disinfection chamber. The processor further controls the air disinfection chamber to disinfect the sucked infected air based on ultraviolet-C (UVC) light and a first valve of the air disinfection chamber to allow passage of the disinfected air out of the air disinfection chamber in a direction opposite to a direction of movement of the cabin.

In an embodiment, the plurality of sensors may include a set of infrared imaging devices configured to detect thermal changes caused by the direction of sneeze of the user in the cabin. The processor is further configured to determine the occurrence of the sneeze based on the detection of the thermal changes and also determine a location of the user within the cabin by analyzing a thermal signature provided by the infrared sensors.

In an embodiment, the plurality of sensors may further include a set of acoustic sensors configured to detect the sound of the sneeze. The processor is further configured to determine the occurrence of the direction of sneeze based on a combination of the thermal changes and acoustic data from the infrared sensors and the acoustic sensors, and determine the location of the user within the cabin by analyzing the thermal changes provided by the infrared sensors, in conjunction with an intensity and direction of the sound detected by the acoustic sensors.

In an embodiment, the processor is further configured to determine a panel of the set of panels nearest to the determined location of the user in the cabin.

In an embodiment, the processor is further configured to control the set of valves in the determined panel to open the corresponding set of perforations in the determined panel.

In an embodiment, the processor is further configured to determine a direction of the sneeze based on data from the set of infrared sensors and the set of acoustic sensors, wherein the infrared sensors detect thermal movement of air particles in relation to the location of the user, and the acoustic sensors analyze a direction of sound propagation.

In an embodiment, the processor is further configured to control the set of valves to open only the perforations that are located in the direction of the detected sneeze, allowing for the selective suction of the infected air from the specific area towards which the sneeze is directed.

In an embodiment, the processor is further configured to select the set of valves in the nearest panel based on a size and spread of the sneeze detected by the infrared sensors and the acoustic sensors, allowing for precise control over which perforations are open and control the selected set of valves in the nearest panel to open corresponding perforations for suction of the infected air.

In an embodiment, the processor is further configured to control a second valve of the air disinfection chamber to blow the disinfected air back into the cabin.

In an embodiment, the processor is further configured to determine the occurrence of the sneeze and the location of the user sneezing in the cabin using a machine learning model trained by analyzing sneeze patterns detected by the infrared sensors and acoustic sensors, and adjust the valve opening pattern based on historical data and real-time sneeze characteristics.

In an embodiment, the air disinfection chamber further comprises a high-efficiency particulate air (HEPA) filter for capturing airborne particles before exposing the air to UVC light.

In an embodiment, the plurality of sensors may further comprise a set of air quality sensors configured to monitor airborne contaminants in the cabin, and the processor is configured to adjust an intensity of suction by the vacuum box and UVC light exposure based on the monitored airborne contaminants in the cabin.

In an embodiment, the processor is further configured to adjust a suction force of the vacuum box based on a proximity of the user to the perforated panels, as detected by proximity sensors, in order to minimize discomfort.

In an embodiment, the processor is further configured to adjust the disinfection process based on a movement status of the elevator, intensifying the disinfection during elevator stops and minimizing activity during movement.

In an embodiment, the processor is further configured to operate in an energy-efficient mode, reducing suction and UV light intensity when the elevator is unoccupied or lightly used.

### BRIEF DESCRIPTION OF THE DRAWINGS

The present application can be best understood by reference to the following description taken in conjunction with the accompanying drawing figures, in which like parts may be referred to by like numerals.

FIG. 1 illustrates a scenario diagram 100 of an elevator air disinfection system, in accordance with an embodiment of the present disclosure.

FIG. 2 illustrates a block diagram 200 of an air disinfection system of FIG. 1, in accordance with an embodiment of the present disclosure.

FIG. 3 illustrates an exemplary scenario diagram 300 of a cabin of an elevator integrated with the air disinfection system 102 of the FIGS. 1 and 2, in accordance with an embodiment of the present disclosure.

FIG. 4 illustrates a structural diagram 400 of an air disinfection system, in accordance with an embodiment of the present disclosure.

### DETAILED DESCRIPTION OF THE DRAWINGS

The following description is presented to enable a person of ordinary skill in the art to make and use the invention and is provided in the context of particular applications and their requirements. Various modifications to the embodiments will be readily apparent to those skilled in the art, and the generic principles defined herein may be applied to other embodiments and applications without departing from the spirit and scope of the invention. Moreover, in the following description, numerous details are set forth for the purpose of explanation. However, one of ordinary skill in the art will realize that the invention might be practiced without the use of these specific details. In other instances, well-known structures and devices are shown in block diagram form in order not to obscure the description of the invention with unnecessary detail. Thus, the invention is not intended to be limited to the embodiments shown, but is to be accorded the widest scope consistent with the principles and features disclosed herein.

While the invention is described in terms of particular examples and illustrative figures, those of ordinary skill in the art will recognize that the invention is not limited to the examples or figures described. Those skilled in the art will recognize that the operations of the various embodiments may be implemented using hardware, software, firmware, or combinations thereof, as appropriate. For example, some processes can be carried out using processors or other digital circuitry under the control of software, firmware, or hard-wired logic. (The term “logic” herein refers to fixed hardware, programmable logic and/or an appropriate combination thereof, as would be recognized by one skilled in the art to carry out the recited functions). Software and firmware can be stored on computer-readable storage media. Some other processes can be implemented using analog circuitry, as is well known to one of ordinary skill in the art. Additionally, memory or other storage, as well as communication components, may be employed in embodiments of the invention.

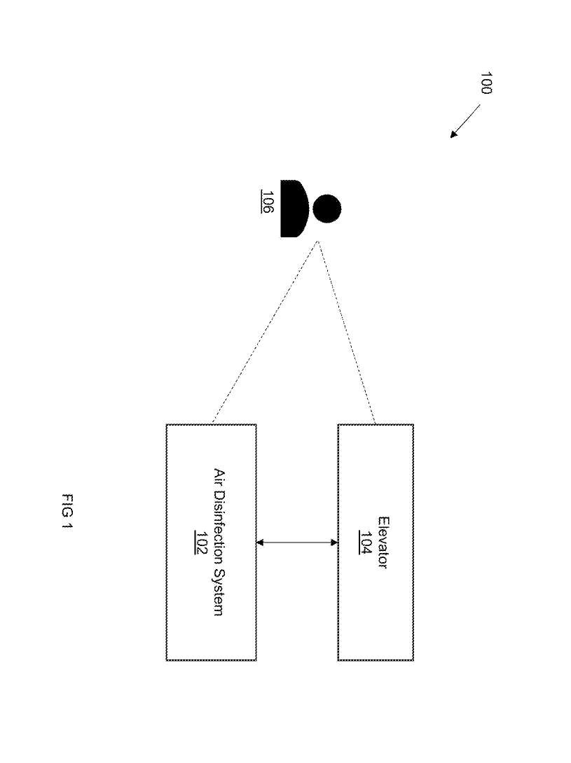

FIG. 1 illustrates a scenario diagram of an elevator air disinfection system, in accordance with an embodiment of the present disclosure. With reference to FIG. 1, there is shown a diagram 100 that includes an air disinfection system 102 and an elevator 104.

In an embodiment, the air disinfection system 102 is integrated into a cabin of the elevator 104. The air disinfection system 102 provides a mechanism for purifying air in enclosed spaces, particularly suited for elevator cabins. The air disinfection system 102 is designed to detect and capture contaminated air in real time, particularly in response to airborne pathogens released through sneezing or coughing. It then subjects the contaminated air to a disinfection process, ensuring the air is purified before being recirculated or expelled from the enclosed space.

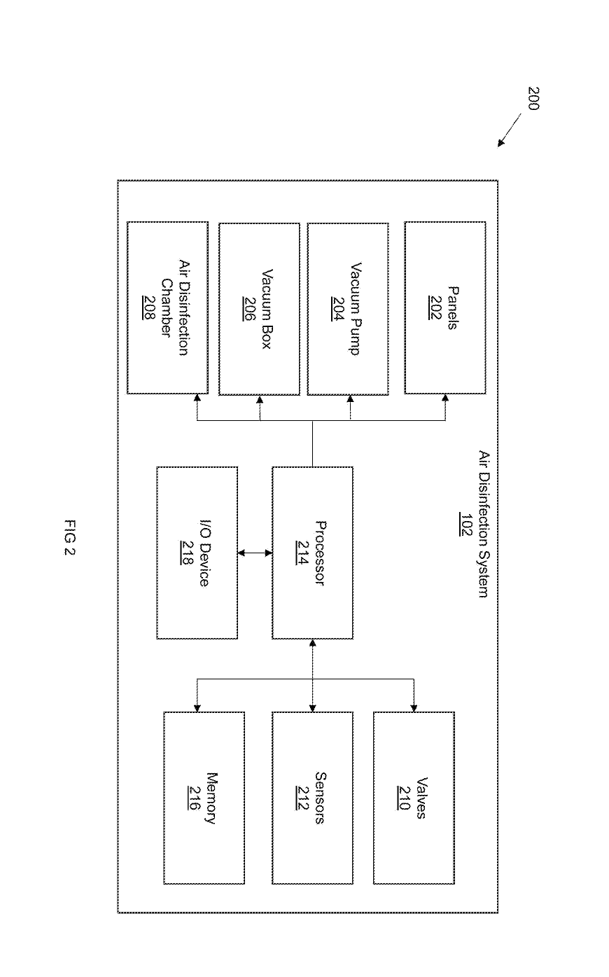

The disinfection process is initiated by a detection mechanism that identifies a source of contamination in the air, such as the release of droplets through a sneeze. Once the contamination is detected, the air disinfection system 102 initiates an airflow control operation, wherein the contaminated air is rapidly drawn away from the localized source. The air is then directed into a specialized air disinfection chamber 208 dedicated for air disinfection, further in FIG. 2, where the contaminated air undergoes a sterilization procedure. In an example, the sterilization process includes, however not limited to, the use of ultraviolet-C (UVC) light. The other sterilization techniques may also include ozone sterilization that utilizes ozone gas to eliminate pathogens by oxidizing their cell membranes. Hydrogen peroxide vapor is another method that effectively disinfects surfaces and air by producing free radicals that disrupt microbial cells. Additionally, ionizing radiation, such as gamma rays, can also be employed for sterilization by damaging the DNA of microorganisms, rendering them inactive. The exposure to the UVC light neutralizes airborne pathogens, rendering the air free of harmful viruses and bacteria.

After the disinfection process, the purified air is either released back into the enclosed space or expelled, depending on the operational requirements. The air disinfection system 102 is capable of responding dynamically to air quality changes and can operate continuously or on-demand, depending on the detected levels of contamination.

The air disinfection system 102 is intended to operate autonomously, requiring minimal human intervention, and is particularly useful in environments such as hospital elevators, where rapid and efficient air purification is essential to preventing the spread of airborne diseases. The core advantage of this system lies in its ability to respond quickly to localized contamination events and ensure that the air within the cabin remains clean and safe for occupants.

The claimed air disinfection system 102 is highly versatile and can be applied in various enclosed spaces where the risk of airborne disease transmission is significant. Primary application areas include hospital elevators, where rapid air purification is critical to minimizing pathogen spread. Additionally, it can be implemented in public transportation systems, such as airplanes, trains, and buses, as well as cruise ship cabins, to ensure cleaner air in confined spaces. Other potential applications include elevators and shared spaces in office buildings, hotels, and other high-traffic environments where maintaining air quality is essential for occupant safety.

Additionally, the technical advantages of an air disinfection system 102 may address key problems in conventional air purification methods. The system's ability to detect and respond to airborne contaminants in real time ensures localized air purification, specifically targeting the source of contamination, such as a sneeze or cough. This targeted approach enhances disinfection efficiency compared to passive systems. The use of UV-C light offers superior pathogen neutralization, effectively addressing the limitation of standard filtration methods. Its adaptability to various enclosed environments further solves the problem of designing a system suitable for diverse spaces without compromising its disinfection capabilities.

FIG. 2 illustrates a block diagram of an air disinfection system 102 of FIG. 1, in accordance with an embodiment of the present disclosure. FIG. 2 provides a detailed block diagram of the air disinfection system 102 illustrating how various internal components interact to purify the air within a confined space, such as the elevator cabin 104. The air disinfection system 102 may include panels 202, a vacuum pump 204, a vacuum box 206, an air disinfection chamber 208, valves 210, sensors 212, a processor 214, a memory 216, an input/output (I/O) device 218.

The panels 202 are integrated into the walls of the cabin 304 and feature numerous perforations. This plurality of perforations, controlled by the air disinfection system 102, allow air to be sucked in from the cabin for disinfection when required. The panels 202 are strategically placed to ensure comprehensive coverage of the cabin area, particularly targeting locations where contaminated air may be concentrated.

In the embodiment, the vacuum pump 204 is crucial for creating the negative pressure necessary to pull the contaminated air from the cabin 104 through the plurality of perforations in the panels 202. The vacuum pump 204 ensures that the airflow is consistent and strong enough to transfer the air efficiently into the next stage of the disinfection process. This air is first held in the vacuum box 206, which temporarily stores the air before passing it into the disinfection chamber 208. The vacuum box 206 in the air disinfection system 102 is a sealed enclosure designed to efficiently extract and sanitize contaminated air from the elevator cabin 104. Its structure typically includes inlet valves 210 that connect to the panels 202 in the cabin 304, allowing air to flow in when activated. Inside, the vacuum box 206 features a HEPA filter that captures larger particles before the air enters a UV-C chamber, where it is exposed to ultraviolet light for disinfection. In an example structural design, the vacuum box 206 has a rectangular or cubic structure made from durable, corrosion-resistant materials such as stainless steel or reinforced plastic, ensuring it remains sealed under pressure. The outer casing includes mounting brackets to securely attach the box within the elevator’s mechanical compartment or hidden panel spaces.

The vacuum pump 204 creates the necessary suction to draw air into the box, ensuring a continuous flow of contaminated air for sterilization. When a sneeze or cough is detected, the processor 214 activates the appropriate valves 210, facilitating the extraction of air, filtration, and subsequent UV-C treatment. This integrated design enhances air quality and minimizes the risk of airborne disease transmission in the enclosed environment of the elevator cabin 104. The vacuum box 206 acts as a buffer, ensuring that the air disinfection system 102 has a steady flow of air for processing, regardless of the demand fluctuations from the cabin environment.

Once the contaminated air is collected in the vacuum box 206, it is transferred to the air disinfection chamber 208. In an embodiment, the air disinfection system 102 includes several essential components: panels 202 with the plurality of perforations in the cabin 304 walls, inlet valves 210 for controlled airflow, and the vacuum box 206 that houses a HEPA filter to capture larger particles before air enters the air disinfection chamber 208. The air disinfection system 102 is equipped with thermal infrared sensors 212 and acoustic sensors 212 to detect sneezes and locate users. The vacuum pump 204 creates the necessary suction to draw in air, while the processor 214 coordinates the operation of all components, ensuring effective air purification and reduced airborne disease transmission within the elevator cabin 104

This air disinfection chamber 208 utilizes a high-efficiency particulate air (HEPA) filter for capturing airborne particles before exposing the air to UVC light. The HEPA filter (High-Efficiency Particulate Air filter) is a type of air filter designed to capture extremely small particles from the air with a high level of efficiency. It is widely used in environments requiring clean air, such as hospitals, airplanes, and laboratories. The filtration process relies on three primary mechanisms: interception, where particles adhere to Fibers; impaction, where larger particles collide with Fibers and get stuck; and diffusion, which captures ultra-small particles through erratic movement caused by molecular collisions. The air disinfection chamber 208 is designed to hold the air long enough for the UV-C light to effectively sterilize it before releasing the cleaned air back into the cabin 304. The air disinfection chamber's 208 structure ensures that all incoming air is thoroughly exposed to the UV-C light, maximizing the disinfection efficiency.

In the embodiment, the valves 210 associated with the perforations in the panels 202 are integral to controlling the airflow in and out of the air disinfection system 102. These valves 210 regulate the opening and closing of the perforations in the panels 202, ensuring that only specific areas of the cabin 304 are targeted for suction. The processor 214 controls the valves 210 based on sensor 212 input, allowing the air disinfection system 102 to react quickly to airborne contaminants like sneezes or coughs.

In the embodiment, the sensors 212, including thermal and acoustic sensors 212, are deployed throughout the cabin to detect airborne contaminants. The air disinfection system 102 employs a combination of thermal infrared sensors 212 and acoustic sensors 212 to enhance sneeze detection and user localization within the cabin. Thermal infrared sensors 212 are designed to detect infrared radiation emitted by objects based on their temperature. Thermal infrared sensors 212 are particularly effective in identifying thermal changes associated with sneezing events. When a person sneezes, the sudden release of warm air creates a distinct thermal signature that these sensors 212 can pick up, allowing the system to determine the presence of a user and locate them within the cabin 304. In the air disinfection system 102, proximity sensors 212 play a crucial role in detecting the presence of users within the elevator cabin 304. These sensors 212 work by emitting signals and measuring the time it takes for the signals to return after reflecting off nearby objects, thus determining the distance to the user. When a sneeze or cough is detected by thermal infrared or acoustic sensors 212, the proximity sensors 212 help confirm user location and proximity to the set of panels 202. All sensors 212 are linked to a central processor 214, which integrates data from the thermal infrared sensors 212 that detect thermal changes, acoustic sensors 212 that capture sneeze sounds, and proximity sensors 212 that assess user distance. This coordinated approach allows the system to accurately identify the location and presence of potential contaminants, activating the nearest suction panels 202 for effective air purification while ensuring minimal inconvenience to passengers.

In addition to thermal sensors 212, the system utilizes acoustic sensors 212 that are specifically configured to detect sound waves, including the distinct sound of a sneeze. Acoustic sensors 212 capture audio data, analysing sound intensity and frequency patterns. By processing the sound data, the acoustic sensors 212 confirm the occurrence of a sneeze and provide information about its intensity and direction. This capability is crucial for accurately identifying the location of the user in relation to the sensors 212. The integration of both sensor 212 types allows the processor 214 to determine not only the occurrence of a sneeze but also the precise location of the user within the elevator cabin 104. This combined approach enhances the overall efficacy of the air disinfection system 102, ensuring that nearby suction panels 202 are activated to maximize air purification and reduce the risk of airborne disease transmission.

In the embodiment, the processor 214 coordinates the operation of all components. It processes data from the sensors 212 controls the valves 210, manages the vacuum pump 204, and regulates the disinfection chamber 208. The processor 214 is programmed to respond dynamically to various scenarios within the cabin 304, ensuring that the air disinfection system 102 can adapt to different levels of contamination and airflow requirements. It also runs algorithms that allow the air disinfection system 102 to learn from previous events and optimize its performance over time.

The processor 214 may include suitable logic, circuitry, and interfaces that may be configured to execute program instructions associated with a set of operations to be executed to determine weight distribution, provide the output signal or control the speaker, the display screen, or the haptic device. The processor 214 may include one or more processing units, which may be implemented as an integrated processor or a cluster of processors that perform the functions of the one or more processing units, collectively. The processor 214 may be implemented based on a number of processor technologies known in the art. Example implementations of the processor 214 may include, but are not limited to, an x86-based processor, a Graphics Processing Unit (GPU), a Reduced Instruction Set Computing (RISC) processor, an Application-Specific Integrated Circuit (ASIC) processor, a Complex Instruction Set Computing (CISC) processor, a microcontroller, a central processing unit (CPU), and/or other computing circuits.

Further in an embodiment, the memory 216 component stores all operational data, including sensor 212 readings, system performance metrics, and historical usage information. A suitable Machine Learning model for analysing historical data in the air disinfection system 102 is the Random Forest classifier. This ensemble learning method is effective for classification tasks, such as predicting the likelihood of a sneeze based on various sensor readings (thermal, acoustic, and proximity data). It can handle a mix of continuous and categorical variables, making it robust for real-time data integration from multiple sensors 212. Additionally, Random Forest can manage noisy data and is less prone to overfitting compared to other models, ensuring reliable predictions in dynamic environments like an elevator cabin 104. This model can help optimize the response of the system by learning patterns in user behaviour and sneeze detection, improving overall air purification efficiency. This data allows the system to adjust its functionality based on prior events, enhancing the effectiveness of the disinfection process. The memory 216 also holds important configuration settings and system logs for maintenance and troubleshooting purposes.

The air disinfection system 102 uses a machine learning model to accurately detect the occurrence of a sneeze and pinpoint the location of the user in the cabin 304. This is achieved by analyzing data collected from infrared imaging devices and acoustic sensors 212. The infrared imaging devices detect thermal changes caused by the sneeze, while the acoustic sensors 212 capture the sound and direction of the sneeze.

The machine learning model is trained using historical data of sneeze events, which include various sneeze patterns, directions, intensities, and corresponding user locations. The model is continuously improved by analyzing real-time data from the sensors 212 in the cabin. The processor 214 uses this trained model to detect sneezes in real-time and determine the exact direction and spread of the sneeze particles based on the combination of thermal and acoustic data.

When a sneeze is detected, the processor 214 references both historical patterns and real-time sneeze characteristics to adjust the valve opening pattern. This process ensures that only the valves 210 nearest to the sneeze are opened, maximizing the suction force and minimizing discomfort for other passengers. The model takes into account factors such as the direction of the sneeze, intensity, proximity to panels 202, and the likely spread of particles based on previous sneeze data.

To further enhance performance, the system regularly updates the machine learning model by incorporating new sneeze data and real-time adjustments made during previous operations. This enables the system to become more effective over time, making intelligent decisions about which specific perforations in a panel 202 should be opened to focus the suction on the sneeze particles and reduce unnecessary suction in other areas of the cabin 304.

Finally, the I/O device 218 provides a user interface for air disinfection system 102 monitoring and control. Through this device, users or maintenance personnel can access system diagnostics, view air quality data, and adjust settings such as UV-C light intensity or suction force. The interface ensures that the air disinfection system 102 remains user-friendly and allows for real-time adjustments, ensuring that the air disinfection system 102 can be tailored to the needs of the specific environment. In an embodiment, the processor 214 may determine the status of the disinfection process and display the determined status on the I/O device 218.

The I/O device 218 may include suitable logic, circuitry, interfaces, and/or code that may be configured to receive an input and provide an output based on the received input. For example, the I/O device 218 may receive a first user input indicative of a request to convert the source audio. The first user input may include the source audio. For example, the I/O device 218 may include a microphone through which the user may record a voice of the user as the source audio. Alternatively, the source audio may be selected by the user from a set of audio files stored on the electronic device. The I/O device 218 may be further configured to render the converted audio. Examples of the I/O device 206 may include, but are not limited to, a display (e.g., a touch screen), a keyboard, a mouse, a joystick, a microphone, or a speaker. Examples of the I/O device 218 may further include braille I/O devices, such as, braille keyboards and braille readers.

The coordinated functioning of these components ensures that the air disinfection system can efficiently detect, capture, and sterilize contaminated air in enclosed spaces, providing a healthier environment for occupants. Each component plays a specific role in ensuring the system operates smoothly and effectively, solving the problem of airborne contamination in a dynamic and adaptable manner.

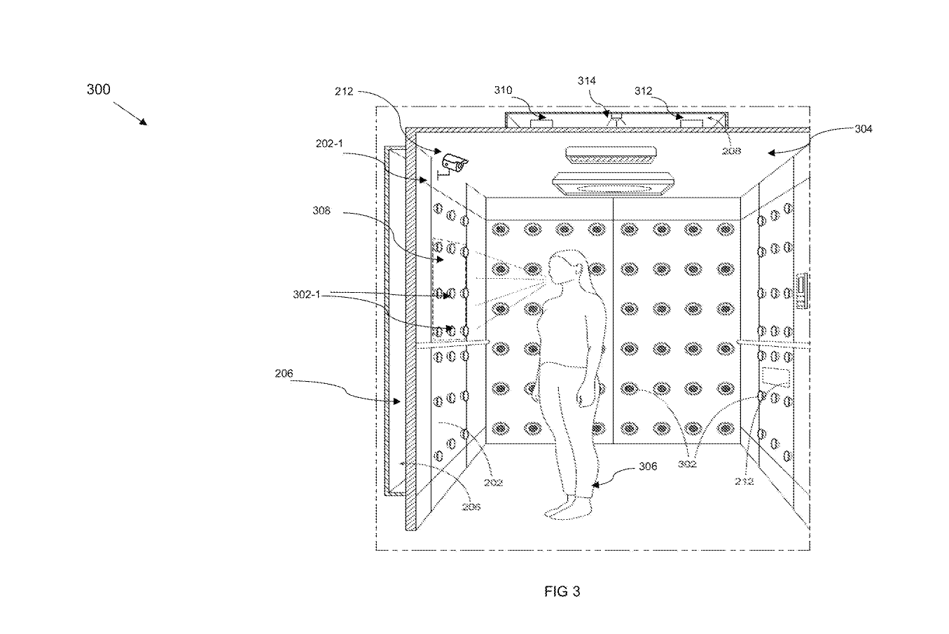

FIG. 3 illustrates an example scenario of a cabin of an elevator integrated with the air disinfection system 102 of FIGS. 1 and 2, in accordance with an embodiment of the present disclosure.

In an example scenario, when a user 306 enters the elevator cabin 304 and sneezes, a series of events is triggered, starting with the detection of the sneeze by the sensors 212 within the air disinfection system 102. These sensors 212 (comprising thermal infrared sensors and acoustic sensors) are strategically placed around the cabin to monitor the environment for changes in temperature and sound. The thermal infrared sensors 212 detect the rapid expulsion of warm air from the sneeze, while the acoustic sensors 212 capture the sound and intensity of the sneeze, precisely determining the direction and area affected by the expelled particles.

As illustrated in an example scenario, however not limited to such a scenario only, the user 306 in the cabin 304 sneezes such that the sneezed particles are focused towards a panel 202-1 of the set of panels 202. More specifically, the intensity of sneezed particles is focused towards a portion 308 of the panel 202-1. A set of infrared imaging devices in the sensors 212 detects thermal changes caused by the sneeze of the user 306 in the cabin 304. The processor 214 now determines the occurrence of the sneeze based on the detection of the thermal changes and determine a location of the user 306 within the cabin 304 by analyzing a thermal signature provided by the infrared imaging devices.

In effect, the processor 214 analyses the sensor 212 data to pinpoint the specific location in the cabin 304 where the sneeze occurred. The processor 214 determines the exact direction and spread of the sneeze by interpreting both the thermal changes and the sound propagation. Based on this data, the processor selects the closest panel 202-1 and the specific portion 308 within the cabin 304 that corresponds to the location of the sneeze. Each panel 202 (as defined in claim 1) contains a series of plurality of perforations 302 and associated valves 210, which can be individually controlled to open or close based on the instructions from the processor 214. However, for best results, it is desirable to determine a specific portion of the panel 202-1 which is most affected by the sneeze, for example, the portion 308 as illustrated in FIG. 3.

Once the processor 214 identifies the affected panel 202-1 and the affected portion 308, it controls the valves 210 of a set of plurality of perforations 302-1 lying in the affected portion 308 within the panel 202-1 to selectively open the appropriate perforations that are closest to the direction of the sneeze. This allows for precise and localized intensity of suction of the contaminated air, minimizing the spread of airborne pathogens within the rest of the cabin. The vacuum pump 204 is activated, creating negative pressure inside the vacuum box 206. The opened perforations allow the contaminated air to be sucked into the vacuum box 206, preventing further circulation of the infected air within the elevator cabin 104. For the ease of illustration, the vacuum box 206 is shown to be attached to perforations along the panel 202-1. However, a person of ordinary skilled in the art would appreciate that such vacuum box 206 can be installed along each panel of the set of panels 202.

The contaminated air then moves from the vacuum box 206 to the air disinfection chamber 208, where the real disinfection process takes place. Inside the chamber 208, the infected air is exposed to UV-C light source 314, a powerful disinfectant known for its ability to neutralize bacteria and viruses. The air remains in the disinfection chamber 208 long enough to ensure thorough sanitization, breaking down any harmful particles carried in the air.

Once the air has been disinfected, the processor 214 controls the opening of a first valve 310 within the air disinfection chamber 208, allowing the cleaned air to be released outside the cabin 304. However, this release is carefully controlled to ensure that the disinfected air is directed in the opposite direction to the movement of the cabin 304, preventing recirculation of air in contaminated zones.

The air disinfection chamber 208 may also include a second valve 312 such that the processor 214 controls the opening of a second valve 312 within the air disinfection chamber 208, allowing the cleaned air to be released back inside the cabin 304.

The air disinfection system 102 continues monitoring the cabin 304 even after the air has been disinfected. If any residual contaminants are detected by the sensors 212, the process 214 repeats itself, ensuring that the air within the cabin remains safe and clean for the occupants. The sensors 212 continuously analyze the thermal data and acoustic signals to ensure that no further contamination is present.

This selective suction and disinfection process is a major technical advancement, offering a precise and efficient method of targeting and neutralizing airborne pathogens in a confined space like an elevator 104. The processor's ability to determine the exact size and spread of the sneeze, combined with the sensor's advanced detection capabilities, ensures that only the affected portion of the cabin is targeted for air extraction, resulting in minimal disruption and maximized efficiency.

Through the integration of multiple advanced technologies, including sensors 212 (such as infrared sensors or acoustic sensors), and UV-C disinfection, the system ensures a practical, real-time response to airborne contamination. This makes the system ideal for use in high-risk environments like hospitals, where the rapid spread of airborne diseases is a significant concern.

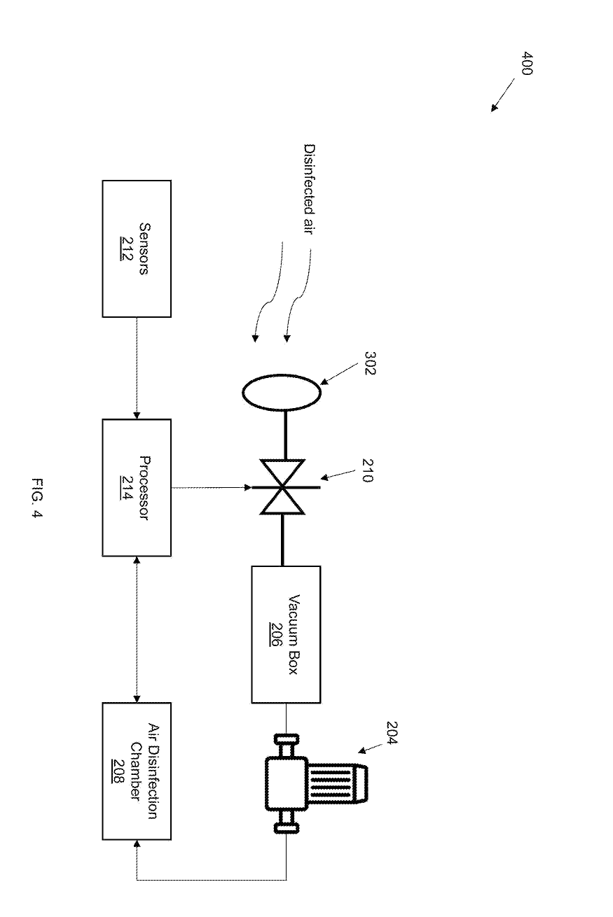

FIG. 4 illustrates a structural diagram of an air disinfection system, in accordance with an embodiment of the present disclosure. In an embodiment, the structural diagram of the air disinfection system 102 illustrates the interconnections and interactions between various internal components essential for efficiently purifying air particles within a confined space, such as an elevator cabin 104. At the core of this air disinfection system 102 is the vacuum pump 204, which plays a pivotal role in creating negative pressure necessary for air movement. When activated, the vacuum pump 204 draws in air from the cabin 304 through the perforated set of panels 202, ensuring that contaminated air is effectively extracted for further processing. This component is engineered for optimal airflow and efficiency, capable of adapting to real-time demands based on contamination levels detected by the air disinfection system 102.

In another embodiment connected to the vacuum pump 204 is the vacuum box 206, which serves as a temporary storage unit for the extracted air. The vacuum box 206 is designed to maintain a steady flow of air, acting as a buffer before the air enters the next stage of disinfection. The vacuum box 206 allows the air disinfection system 102 to efficiently manage fluctuations in airflow and ensures that a consistent volume of air is available for disinfection. The combination of the valves 210, vacuum pump 204, and vacuum box 206 effectively prevents backflow, maintaining a one-way flow of air toward the air disinfection chamber 208.

Once the air is collected in the vacuum box 208, it is directed to the air disinfection chamber 208. The air disinfection chamber 208 is the heart of the disinfection process, utilizing UV-C light technology to neutralize pathogens present in the contaminated air. The structural design of the air disinfection chamber maximizes the exposure of the incoming air to UV-C light, ensuring that all airborne viruses and bacteria are effectively sterilized before the air is released back into the cabin. The air disinfection chamber 208 is engineered to facilitate the optimal dwell time of air within it, allowing sufficient time for the disinfection process to occur.

The valves 210 play a crucial role in regulating the airflow between the vacuum box 206, the disinfection chamber 208, and the set of panels 202. These valves 210 are controlled by the processor 214 and can open or close based on real-time inputs from the sensors 212. By selectively allowing or restricting airflow, the valves ensure that only the contaminated air from specific areas of the cabin is processed, enhancing the system's efficiency. The precise control of these valves 210 is essential for targeting air particles extraction and minimizing the potential spread of pathogens.

In some embodiments, the sensors 212 continuously monitor the cabin environment for indicators of airborne contamination. These sensors 212, which include thermal and acoustic sensors 212, provide real-time data to the processor 214 regarding the presence of sneezes or coughs. By detecting changes in temperature and sound, the sensors 212 enable the air disinfection system 102 to quickly identify the location of contaminants, allowing for immediate activation of the relevant valves 210 and a set of panels 202. This rapid response capability is vital for minimizing the exposure of occupants to airborne pathogens.

At the center of this operational synergy is the processor 214, which serves as the control unit for the entire air disinfection system 102. The processor 214 receives input from the sensors 212 and processes this information to make real-time decisions regarding the operation of the vacuum pump 204, valves 210, and air disinfection chamber 208. It coordinates the actions of all components, ensuring that the system operates efficiently and effectively in response to contamination events. The processor 214 also utilizes algorithms to learn from historical data, continually optimizing the system’s performance based on usage patterns and environmental conditions.

In the above view of, the interconnected components within the structural diagram of the air disinfection system work harmoniously to detect, extract, and disinfect contaminated air particles in enclosed spaces. The collaborative functionality of the vacuum pump 204, vacuum box 206, valves 210, air disinfection chamber 208, sensors 212, and processor 214 ensures a responsive and efficient air disinfection system 102 capable of maintaining a healthier environment for occupants in real-time. Each component contributes to the overall effectiveness of the air disinfection process, providing a robust solution to combat airborne disease transmission.

What is claimed is:

1. An air disinfection system for disinfecting a cabin of an elevator, comprising:
a set of panels, wherein each panel of the set of panels is mounted on a corresponding wall of a set of walls of the cabin;
a vacuum box;
a vacuum pump configured to create negative pressure in the vacuum box;
an air disinfection chamber;
a plurality of perforations in each panel;
a plurality of valves in each panel, wherein each valve of the plurality of valves controls opening and closing of a corresponding perforation of the plurality of perforations;
a plurality of sensors configured to detect a sneeze of a user in the cabin; and
a processor configured to:
control, based on the detection of the sneeze of the user in a specific portion of the cabin, a set of valves of the plurality of valves to open a corresponding set of perforations of the plurality of perforations, wherein the set of perforations are within the specific portion of the cabin in which sneeze is detected;
control the vacuum box to create negative pressure to suck infected air from the specific portion of cabin via the opened set of perforations, wherein the sucked infected air is transferred to the air disinfection chamber;
control the air disinfection chamber to disinfect the sucked infected air based on ultraviolet-C (UVC) light; and
control a first valve of the air disinfection chamber to allow passage of the disinfected air out of the air disinfection chamber in a direction opposite to a direction of movement of the cabin.

2. The air disinfection system according to claim 1, wherein
the plurality of sensors includes a set of infrared imaging devices configured to detect thermal changes caused by the sneeze of the user in the cabin, and
the processor is further configured to:
determine the occurrence of the sneeze based on the detection of the thermal changes; and
determine a location of the user within the cabin by analysing a thermal signature provided by the infrared imaging devices.

3. The air disinfection system according to claim 2, wherein
the plurality of sensors further includes a set of acoustic sensors configured to detect the sound of the sneeze, and
the processor is further configured to:
determine the occurrence of the sneeze based on a combination of the thermal changes and acoustic data from the infrared imaging devices and the acoustic sensors, and
determine the location of the user within the cabin by analysing the thermal changes provided by the infrared imaging devices, in conjunction with an intensity and direction of the sound detected by the acoustic sensors.

4. The air disinfection system according to claim 3, wherein the processor is further configured to determine a panel of the set of panels nearest to the determined location of the user in the cabin.

5. The air disinfection system according to claim 4, wherein the processor is further configured to control the set of valves in the determined panel to open the corresponding set of perforations in the determined panel.

6. The air disinfection system according to claim 2, wherein the processor is further configured to determine a direction of the sneeze based on data from the set of infrared imaging devices and the set of acoustic sensors, wherein the infrared imaging devices detect thermal movement of air particles in relation to the location of the user, and the acoustic sensors analyse a direction of sound propagation.

7. The air disinfection system according to claim 6, wherein the processor is further configured to control the set of valves to open only the perforations that are located in the direction of the detected sneeze, allowing for the selective suction of the infected air from the specific area towards which the sneeze is directed.

8. The air disinfection system according to claim 4, wherein the processor is further configured to:
select the set of valves in the nearest panel based on a size and spread of the sneeze detected by the infrared imaging devices and the acoustic sensors, allowing for precise control over which perforations are opened; and
control the selected set of valves in the nearest panel to open corresponding perforations for suction of the infected air.

9. The air disinfection system according to claim 1, wherein the processor is further configured to control a second valve of the air disinfection chamber to blow the disinfected air back into the cabin.

10. The air disinfection system according to claim 3, wherein the processor is further configured to determine the occurrence of the sneeze and the location of the user sneezing in the cabin using a machine learning model trained by analysing direction of sneeze patterns detected by the infrared imaging devices and acoustic sensors, and adjust the valve opening pattern based on historical data and real-time sneeze characteristics.

11. The air disinfection system according to claim 1, wherein the air disinfection chamber further comprises a high-efficiency particulate air (HEPA) filter for capturing airborne particles before exposing the air to UVC light.

12. The air disinfection system according to claim 1, the plurality of sensors comprises a set of air quality sensors configured to monitor airborne contaminants in the cabin, and the processor is configured to adjust an intensity of suction by the vacuum box and UVC light exposure based on the monitored airborne contaminants in the cabin.

13. The air disinfection system according to claim 1, wherein the processor is further configured to adjust a suction force of the vacuum box based on a proximity of the user to the perforated panels, as detected by proximity sensors, in order to minimize discomfort.

14. The air disinfection system according to claim 1, wherein the processor is configured to adjust the disinfection process based on a movement status of the elevator, intensifying the disinfection during elevator stops and minimizing activity during movement.

15. The air disinfection system according to claim 1, wherein the processor is further configured to operate in an energy-efficient mode, reducing suction and UV light intensity when the elevator is unoccupied or lightly used.
